(19) United States
(12) Patent Application Publication
(10) Pub. No.: US 2011/0092532 A1
(43) Pub. Date: Apr. 21, 2011

(54) APOMORPHINE PHARMACEUTICAL DOSAGE SECURITY SYSTEM

(76) Inventor: John Todhunter, Delaplane, VA (US)

(21) Appl. No.: 12/906,870

(22) Filed: Oct. 18, 2010

Related U.S. Application Data
(60) Provisional application No. 61/252,494, filed on Oct. 16, 2009.

(51) Int. Cl. A61K 31/435

(52) U.S. Cl. 514/284

(57) ABSTRACT

An apomorphine pharmaceutical dosage security system embodied in multiple dosage forms including oral dosage forms, transdermal delivery patches, nasal delivery forms, and transdermal dosage forms. Within the present invention is an amount of apomorphine adapted to be bioavailable to a degree sufficient to cause emesis or severe nausea when an overdose amount of a therapeutic agent enters systemic circulation.

### RELATED APPLICATIONS

The present application claims priority from U.S. Provisional Patent Application Ser. No. 61/252,494 titled Apomorphine Pharmaceutical Dosage Security System, filed Oct. 16, 2009, which is incorporated herein by reference.

### FIELD OF THE INVENTION

The present invention relates to the field of secured pharmaceutical devices and more specifically to the field of pharmaceutical dosages having therapeutic agents coupled with emetics.

### BACKGROUND

Alexandre Dumas in the The Count of Monte Cristo reminded the world that “in medicine use is made of the most violent poisons, which become, according as they are employed, most salutary remedies.” The very same compositions, which in specific doses heal, damage the human body when taken under non-medicinal conditions. Unfortunately, compositions intended as medicinal frequently are used in ways unintended by the prescribing physician. It is often the case that drugs offer a euphoria in addition to their medicinal qualities, which lures patients with more ingenuity than foresight into altering their medication for purposes of entertainment or relaxation. Taking medicine for purposes other than its inherent healing properties is a sure sign of medicinal abuse, and medicinal abuse is becoming more common.

The healthcare provider is faced with multiple avenues for injecting drugs into the bloodstream: pills; injection; intravenous delivery devices; and transdermal delivery systems. Among the forms of medicine delivery, pills are perhaps the most easily abused. Pills are generally administered in large quantities and are meant to be spaced over long periods of time. It is typically a mere warning label that stands between the patient and pill dosage alteration. For this reason, doctors often prefer alternate drug delivery methods for drugs that are commonly abused, for example opioids. The transdermal delivery devices, commonly known simply as “patches,” are the current preferred method for delivering addictive agents into the bloodstream.

Even patches are not entirely abuse resistant. Patents and patent applications directed to foiling the abuser of medicinal patches exist; e.g. United States Published Patent Applications No. 2002/0106329; 2007/0065365; 2007/0065364; 2007/0048228; 2007/0003616; 2006/0193782; 2006/0188447; 2006/0039864; 2006/0002860; 2006/0002859; 2005/0236741; 2005/0214223; 2005/0236741; 2005/0186139; 2006/019881; and 2004/0126323. A current method for preventing patch abuse includes positioning an emetic within the patch that in certain abuse situations triggers emesis within the abusing individual. Current abuse-preventing patches do include an inadequacy: overestimating the sophistication of the abuser.

Taking U.S. Published Patent Application No. 2007/0065365 as an example, it purports to disclose an abuse-resistant transdermal system which contains, in addition to one or more active ingredients with potential for abuse, at least one gel-forming agent in quantities such that it forms a gel with a minimum quantity of an aqueous liquid, and contains as further agents which complicate or prevent abuse at least one emetic, and/or at least one dye as an aversive agent. The '365 published application teaches the use of apomorphine as an emetic, yet only in amounts of between 3.0 mg to 7.0 mg. This cautious use of apomorphine as an emetic ensures emesis during complicated drug diversions, such as parenteral injection; but generally neglects to produce emesis in other diversion actions. While the sophisticated abuser of therapeutic agents within a patch may take the time and effort to disassemble a patch to inject or snort its contents, the less sophisticated abuser may opt for the simpler methods of simply swallowing the patch or tossing the patch into a liquid and drinking the liquid.

Apomorphine has been used a means for securing against complicated diversions in patches and pills. U.S. Published Patent Application No. 2003/0170181 purports to disclose a method for treating a patient with a methylphenidate-responsive condition that is at a risk of abusing or becoming addicted to methylphenidate. The patient is first evaluated for an elevated risk of drug abuse or addiction through psychological evaluations and then treated with a methylphenidate product that includes an emesis-inducing agent that is inert when ingested orally and only produces emesis when snorted or taken intravenously or a topical analgesic that is inert when ingested orally and only produces irritation when snorted or taken intravenously. The method includes delivering the methylphenidate in a pulsatile delivery system such that the emesis-inducing agent or the topical analgesic is included in one of the pulsatile dosages. The '181 application discloses the use of apomorphine as an emetic in pills and capsules, but again, only in quantities capable of producing emesis in high-efficacy diversion techniques. Indeed, this application goes on to stress that the amount of apomorphine must never exceed 10 mg in a total dosage form.

Apomorphine has side effects that include nausea and emesis, depending on the levels of apomorphine effectively absorbed by the body. The effect of apomorphine on an individual varies significantly with its route of introduction into the body. Apomorphine is not effectively absorbed through the skin and is rapidly inactivated when ingested orally. However, apomorphine is effectively bioavailable in amounts comparable to the initially administered amount when taken up through the membranes of the nasal passages, sublingually, transbucally, or injected intravenously or subcutaneously. The loss of bioavailability after oral ingestion is due to “first pass” metabolism in the liver. The other above-mentioned routes do not encounter the chemical deactivation mechanisms of the liver, which accounts for the differential bioavailability of apomorphine when comparing oral to nasal, transbuccal, or injection routes. While weakly bioavailable when administered orally, apomorphine bioavailability is generally non-existent when administered transdermally due to apomorphine's inability to cross intact skin in any meaningful manner.

Campbell et al. (1980) have demonstrated that tissue levels of apomorphine are not detectable in rats after oral ingestion unless the portal vein (which provides direct flow to the liver from the stomach and intestines and enables “first pass” metabolism) is shunted to preclude first pass metabolism. Campbell A, Kula N S, Jeppsson B and Baldessarini J (1980) “Oral bioavailability of apomorphine in the rat with a portocaval venous anastomosis”, Eur. J. Pharmacol., 67(1): 139-142. Gancher et al. have studied the absorption and clinical effect of sublingual (SL) and transdermal apomorphine in parkinsonism. Gancher S T, Nutt J G and Woodward W R (1991) “Absorption of apomorphine by various routes in Parkinsonism”, Movement Disorders, 6(3): 212-216 Patients received single SL apomorphine doses (N=7) and the absorption was compared with parenteral [i.e.: injection] (N=5) and oral (N=4) doses. One patient received a transdermal dose of apomorphine. The relative bioavailability of SL apomorphine was reported as follows: parenteral injection, 100%; sublingual, 10%-22%; oral, <4.0%; and transdermal, not detectable.

In the Gancher study, three patients with motor fluctuations responded to SL apomorphine, with latency to effect of 20-40 min and a duration of effect of 15-100 min. One patient used SL apomorphine as an adjunct with levodopa, and during one month reported a large decrease in “off” periods. The authors concluded that apomorphine is effectively absorbed by the sublingual route.

It should be noted that by the sublingual route a significant, but unknown, proportion of the administered dose will be lost to oral ingestion due to admixture with the saliva secreted continuously from the sublingual salivary glands and which is continuously swallowed in normal individuals. This suggests that intranasal uptake of apomorphine should be much more efficient than sublingual uptake, and indeed, this is demonstrated by further studies.

The high effectiveness of intranasal uptake of apomorphine has been demonstrated by Sam et al. (1995). Sam E, Jeanjean A P, Maloteaux J M and Verbeke M (1995) “Apomorphine pharmacokinetics in parkinsonism after intranasal and subcutaneous application.”, Eur. J. Drug Metabol. Pharmacokinetics, 20(1): 27-33. These investigators administered apomorphine subcutaneously and intra-nasally to 7 patients suffering from Parkinsonism with ‘on-off’ problems. This comparative pharmacokinetic study showed that the two routes of administration are comparable with respect to absorption kinetics. Apomorphine is rapidly absorbed when administered intra-nasally or subcutaneously with an absorption half life of 8.6 min and 5.8 min, respectively. The high rate of absorption is also reflected by the time for the plasma concentration to peak (Tmax) and the lag times reported in this study. The Tmax was 23 min for intranasal route and 18 min for the subcutaneous route while the lag times were 2.8 min and 3.9 min, respectively. The bioavailability of intranasal apomorphine compared to the subcutaneous route amounted to 45%. After intranasal and subcutaneous administrations, the elimination half life of apomorphine amounted to 31 min and 27 min, respectively.

Apomorphine has also been used as a model emetic in studies for screening of anti-emetic drugs as well as in emergency medicine to induce vomiting to clear the stomach in cases of accidental ingestion of non-caustic and non-corrosive poisons as an alternative to gastric lavage.

Hvarfner et al. (1995) have demonstrated that the amount of intravenous apomorphine which will reliably induce emesis in healthy volunteers is 2.5 mg+0.5 mg in the absence of counteracting, e.g. anti-emetic, drugs. Hvarfner A, Hammas B, Thorn S-E, and Wattwil, M. (1995) “The influence of propafol on vomiting induced by apomorphine”, Anesth. Analges., 80: 967-969. This study involved ten male volunteers aged 23-39. Accordingly, a reliable and effective emetic amount of apomorphine is known.

Notwithstanding the human body's predisposition to emesis on contact with significant amounts of apomorphine, organisms tend to be generally tolerant of apomorphine with regard to toxicity levels. According to data reported in the Hazardous Substances Data Base, a part of the Federal TOXNET data base, the following LD₅₀ (median lethal dose) and toxic dose values are available and shown in Table 1.

TABLE 1

| Subject | Route | Toxicity | |
|---|---|---|---|
| Mouse | Oral | 300 | mg/Kg, LD₅₀ |
| Mouse | Intra-peritoneal | 160 | mg/Kg, LD₅₀ |
| Mouse | Intra-venous | 56 | mg/Kg, LD₅₀ |

| | | |
|---|---|---|
| Dog | Reversible Toxicity | 36-71 mg/Kg |
| | Seen | (subcutaneous) |
| Cat | Reversible Toxicity | 136 mg/Kg |
| | Seen | (subcutaneous) |

As shown by Table 1, the ability of an organism to withstand significant quantities of apomorphine allows apomorphine a high degree of safety, particularly when compared to the amounts of apomorphine necessary for effective emesis.

Therefore, there is a need for pharmaceutical security system capable of universal bioavailability. In other words, the amount of emetic within the pharmaceutical carrier should include an amount of apomorphine capable of triggering emesis in a human user in all forms of diversion, particularly injection, swallowing, smoking, and intranasal and sublingual delivery. There is further need of an emetic having an inert chemistry that allows it to intermingle with a therapeutic agent, thereby obviating the need for intra-carrier separation structure between the emetic and the therapeutic agent.

### SUMMARY

The present invention is directed to an apomorphine pharmaceutical dosage security system. The present invention includes many aspects, each related to the differential bioavailability characteristics of apomorphine. An oral dosage form of the present invention includes an oral carrier, which may include a capsule, pill, or other oral delivery vehicle existing in the pharmaceutical arts. The oral carrier includes a frequently-abused therapeutic agent. The therapeutic agent is preferably one with an overdose amount known with relative certainty. Within the oral carrier is an amount of apomorphine disbursed among the therapeutic agent. The amount of apomorphine per oral dosage is determined by first determining an overdose amount of the therapeutic agent. The amount of apomorphine effective to produce a universal bioavailability of a particular value is then divided substantially evenly among the number of pills required to meet or surpass the overdose amount. A preferred embodiment of the oral dosage form includes an amount of apomorphine of at least 50.0 mg divided into the number of oral dosage forms required to meet or surpass the overdose amount, or enough apomorphine to allow a universal bioavailability of 2.0 mg of apomorphine divided into the number of oral dosage forms required to meet or surpass the overdose amount. Other embodiments may include an amount of apomorphine adapted to allow a universal bioavailability of at least 2.5 mg of apomorphine divided into the number of oral dosage forms required to meet or surpass the overdose amount, or at least 67.5 mg of apomorphine divided into the number of oral dosage forms required to meet or surpass the overdose amount.

Universal bioavailability is the amount of apomorphine available for systemic circulation by the body irrespective of the route of apomorphine exposure—except via skin absorption. For practical purposes, apomorphine is generally incapable of permeating skin, and is thus skin absorption is not included in the definition of universal bioavailability. Apomorphine is, however, substantially available for systemic circulation through the routes of injection (e.g. intravenous, subcutaneous, and the like), sublingual administration, intranasal administration, and oral administration. Each exposure route possesses a different rate of availability of apomorphine due to various mechanisms within the body; for example, oral administration of apomorphine subjects the apomorphine to “first pass” metabolism of the liver, which drastically diminishes the amount of apomorphine available to the body for processing. When a universally bioavailable amount is stated herein, it is meant that at least that amount would be available to the body for systemic uptake irrespective of the initial amount offered to the body and irrespective of whether the exposure route included injection, sublingual administration, intranasal administration, or oral administration.

The present invention further includes a system of oral dosage forms that includes multiple oral dosage forms. Within each oral dosage form is an amount of the therapeutic agent. An overdose amount of the therapeutic agent and the number of dosages forms necessary to meet or surpass that overdose amount is determined; and a security amount of apomorphine is disbursed within that number of dosage forms. The security amount is preferably at least 50.0 mg of apomorphine, or an amount of apomorphine adapted to have a universal bioavailability of at least 2.0 mg. Other embodiments may include an amount of apomorphine adapted to allow a universal bioavailability of at least 2.5 mg, or at least 67.5 mg of apomorphine.

The present invention further includes a transdermal delivery system. The transdermal delivery system includes a therapeutic composition stratum and at least 50.0 mg of apomorphine disbursed within the stratum, or an amount of apomorphine adapted to allow a universal bioavailability of at least 2.0 mg of apomorphine.

The present invention further includes a nasal dosage system. The nasal dosage system includes a nasal dosage form, such as an aqueous nose spray, having a therapeutic agent. The therapeutic agent has a predetermined overdose amount, and the volume containing that predetermined overdose amount also preferably contains at least 2 mg of apomorphine, or an amount of apomorphine adapted to generate an intranasal bioavailability of at least 1.0 mg of apomorphine.

The present invention also includes a topical cream with a transdermal dosage form. Within the transdermal dosage form is a therapeutic agent with a predetermined overdose amount. The volume of the transdermal dosage form containing the predetermined overdose amount also preferably includes at least 50.0 mg of apomorphine disbursed therein, or an amount of apomorphine adapted to allow a universal bioavailability of at least 2.0 mg of apomorphine.

Therefore, it is an aspect of the present invention to allow a safe pharmaceutical security system.

It is a further aspect of the present invention to allow a security system that is inert when the pharmaceutical product is used as intended.

It is a further aspect of the present invention to allow a security system that reliably induces nausea or emesis in a substantial portion of the population when the pharmaceutical product is used in an abusive manner.

It is a further aspect of the present invention to allow a security system capable of reliably producing emesis in an over-dosing condition for oral formulations prior to absorption of an amount of the pharmaceutical product necessary to produce adverse health effects.

It is a further aspect of the present invention to allow a security system that in use minimizes harm directly related to the mechanism of the security system.

These aspects of the invention are not meant to be exclusive. Furthermore, some features may apply to certain versions of the invention, but not others. Other features, aspects, and advantages of the present invention will be readily apparent to those of ordinary skill in the art when read in conjunction with the following description, and accompanying drawings.

### BRIEF DESCRIPTION OF THE DRAWINGS

FIG. 1 is an isometric view of an oral dosage form of the present invention.

FIG. 2 is an isometric view of an oral dosage form of the present invention.

FIG. 3 is a top plan view of an oral dosage system of the present invention.

FIG. 4 is a side, cutaway view of an oral dosage form of the present invention.

FIG. 5 is a side, cutaway view of an oral dosage form of the present invention.

FIG. 6 is a perspective view of an oral dosage form of the present invention.

FIG. 7 is a perspective view of an oral dosage form of the present invention.

FIG. 8 is a side, cutaway view of a transdermal patch of the present invention.

FIG. 9 is a side, cutaway view of a transdermal patch of the present invention.

### DETAILED DESCRIPTION

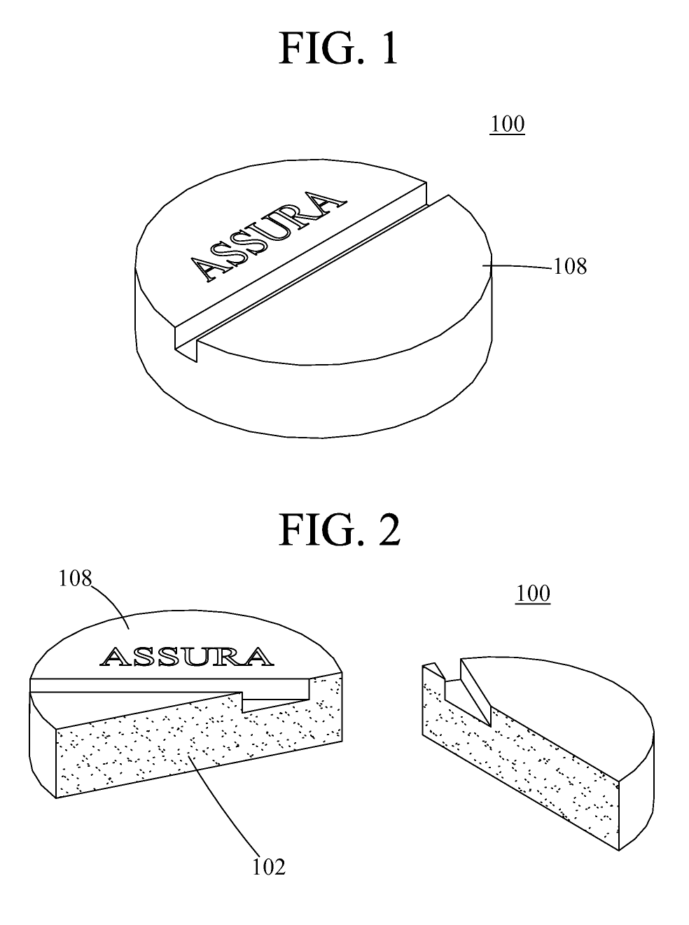

Referring first to FIG. 1, a basic embodiment of the oral dosage form 100 of the present invention is shown. The oral dosage form 100 includes an oral carrier 108 which is the body of the oral dosage form. Versions of the oral carrier 108 may include a tablet form, such as that pictured in FIG. 1. In tablet form the oral carrier 108 includes a combination of a therapeutic agent and apomorphine. As FIG. 2 depicts, the therapeutic agent may be intermingled with the apomorphine.

The therapeutic agent of the present invention includes any chemical administered to an individual for health reasons that when abused is capable of harmful effects. Suitable candidates for the therapeutic agent may include prescription drugs, such as opioids (e.g. oxycodone, meperidine, hydromorphone, hydrocodone, propoxyphene, etc.), morphine, fentanyl, codeine, ketamine, barbiturates, benzodiazepines, and the like; or over-the-counter medications, such as Dextromethorphan. The present invention is generally not limited to any particular therapeutic agent; however, the therapeutic agent should not include a chemical capable of reacting with apomorphine and its derivatives.

Apomorphine is used in conjunction with the therapeutic agent to ensure that a user follows an intended use regimen of the therapeutic agent. The apomorphine of the present invention may include any form of apomorphine, including apomorphine derivatives such as esters, ethers, amides, mixed anhydrides, hemiacetals, glucuronates, sulfates or phosphonates. Apomorphine is a powerful emetic having a varied level of effectiveness, i.e. bioavailability, dependent on its means of introduction into the human body.

There are five primary routes for introducing any therapeutic agent into the human body: by injection; intra-nasally; sublingually; orally; and transdermally. Evidence indicates that the bioavailability of apomorphine, i.e. the ratio of apomorphine administered to the amount of apomorphine processed by the human body to receive the inherent effects of apomorphine, generally varies as shown in Table 2.

TABLE 2

| Route | Relative Bioavailability |
|---|---|
| Injection of Extract | ~100% |
| Intra-nasal | 45% |
| Sublingual | 10%-22% |
| Oral | <4% |
| Transdermal | ~0% |

As the values of Table 2 indicate, transdermal application of apomorphine has allows no significant absorption, while injection of apomorphine allows absorption by the body at a ratio approaching 100%. Certain preferred embodiments of the present invention include amounts of apomorphine adapted to have a universal bioavailability of at least a specific amount. When a universally bioavailable amount is stated herein, it is meant that at least that amount would be available to the body to process irrespective of the initial amount offered to the body and irrespective of whether the exposure route included injection, sublingual administration, intranasal administration, or oral administration. As apomorphine is for all practical purposes incapable of significant skin absorption, apomorphine available from skin absorption is not included in the definition of universal bioavailability. Bioavailability for purposes of the present invention may further be stated in terms of the point of entry of the apomorphine. For example, a sublingual bioavailability amount would indicate the amount of apomorphine available to the body to process, irrespective of the initial amount offered to the body, due to the sublingual absorption of apomorphine.

When administered, apomorphine will begin to produce nauseous effects within an average user at an approximate bioavailability 1.0 mg-2.0 mg. Levels of bioavailable apomorphine beyond 2.0 mg will begin to cause emesis in the average user. A bioavailable amount of approximately 2.5 mg of apomorphine will reliably cause emesis in the average user, and bioavailable amounts of apomorphine greater than 4.0 mg can be expected to cause emesis in approximately 100% of all human users. As Table 2 indicates, however, the introduction routes of apomorphine control the bioavailable amount for a given user.

The present invention spans varying amounts of apomorphine suited to the particular therapeutic agent capable of abuse and the vehicle for supplying it. In the oral dosage form 100, it is preferred that oral dosages having a therapeutic agent frequently abused via oral routes include an amount of apomorphine effective to prevent oral abuse. As the bioavailability of apomorphine via oral ingestion is relatively low, it is preferred that at least 50.0 mg of apomorphine be coupled to the number of oral dosage forms containing an amount of therapeutic agent capable of producing an overdose amount.

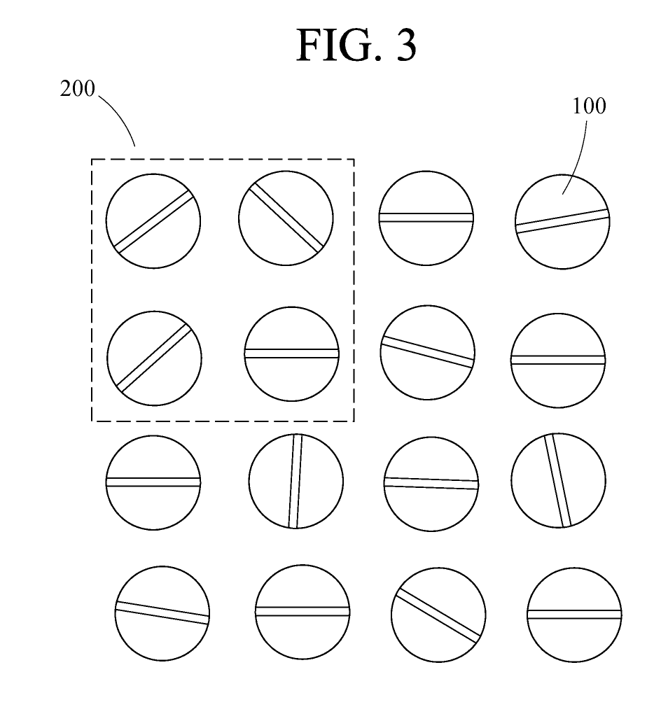

As FIG. 3 shows by way of example, if four oral dosages 100—irrespective of the total number of oral dosages prescribed or dispensed—include an amount of a therapeutic agent possessing an overdose amount, then it is preferred that an oral dosage security system 200 include 50.0 mg of apomorphine distributed within those four oral dosages. Thus, a user that orally ingests the number of oral dosage forms necessary for an overdose of the therapeutic agent, also ingests 50.0 mg of apomorphine, which introduces 2.0 mg of bioavailable apomorphine into the system of the user and will likely lead to emesis. It is preferred that the amount of apomorphine in the oral dosage security system be disbursed substantially equally among the oral dosages therein. In the present example, it is preferred that each oral dosage include approximately 12.5 mg of apomorphine; or in other words, each oral dosage includes a 0.5 mg dose of bioavailable apomorphine.

In guarding against oral overdosing, the user of the present invention is further guarding against all other relevant means of diversion; namely injection, sublingual administration, and intranasal administration. An oral dosage having an amount of apomorphine adapted to cause emesis via oral ingestion necessarily includes an amount of apomorphine adapted to cause emesis in all other relevant forms of diversion, all of which allow a higher degree of bioavailability.

TABLE 3

| Initial | | Injection | | Intra-Nasal | Sublingual | | Oral |
|---|---|---|---|---|---|---|---|
| Apomorphine | | Bioavail- | | Bioavail- | Bioavail- | | (Universal) |
| Amount in | | able | | able | able | | Bioavailable |
| System | | Amount | | Amount | Amount | | Amount |
| 50.0 | mg | 50.0 | mg | 22.5 mg | 5.0-10.0 | mg | 2.0 mg |
| 62.5 | mg | 62.5 | mg | 28.1 mg | 6.3-12.5 | mg | 2.5 mg |
| 75.0 | mg | 75.0 | mg | 33.8 mg | 7.5-14.0 | mg | 3.0 mg |
| 87.5 | mg | 87.5 | mg | 39.4 mg | 8.8-17.0 | mg | 3.5 mg |
| 100.0 | mg | 100.0 | mg | 45.0 mg | 10.0-20.0 | mg | 4.0 mg |
| 112.5 | mg | 112.5 | mg | 50.6 mg | 11.3-22.5 | mg | 4.5 mg |
| 125.0 | mg | 125.0 | mg | 56.25 mg | 12.5-25.0 | mg | 5.0 mg |
| 150.0 | mg | 150.0 | mg | 67.5 mg | 15.0-30.0 | mg | 5.5 mg |

As can be seen in Table 3, an amount that is universally bioavailable at 2.5 mg, will provide an oral bioavailability of 2.5 mg; but also provide a significant amount of bioavailability in the routes of injection, sublingual administration, and intranasal administration; corresponding to 7.5 mg-14.0 mg, 28.1 mg, and 62.5 mg respectively. In all forms of the present invention with a secured apomorphine amount (the amount present in an overdose amount of the therapeutic agent) having a universal bioavailability of at least 2.5 mg, substantially greater amounts of apomorphine will be available to the body via other diversion means. For example, a secured amount of apomorphine of 50.0 mg will be capable of causing emesis in a substantial portion of the population due to an oral bioavailability of 2.0 mg, but that same quantity of apomorphine will have a similar bioavailability of 22.5 mg intra-nasally, which will produce emesis is approximately 100% of the human population. With apomorphine, to guard against oral abuse can guard against all abuse.

Other preferred embodiments of the oral dosage security system 200 will include greater amounts of apomorphine. A bioavailable amount of apomorphine of 2.0 mg bioavailable apomorphine is regarded as the minimum in ensuring emesis in a substantial portion of a population. It is preferred that the present invention be adapted to deliver at least 2.5 mg bioavailable apomorphine, and even more preferably 3.0 mg bioavailable apomorphine, in the number of dosage forms required to reach an overdose amount of therapeutic agent.

The system of present invention is preferably adapted to comprise oral dosages encompassing a concentration of a therapeutic agent employed for recreational effects (e.g. to obtain a high) rather than intentional harmful effects (e.g. suicide). The difference between recreational effects and harmful effects lies in the number or oral dosages in which an abuser would indulge. Whereas a user seeking to initiate harm might haphazardly ingest an arbitrary mass of pills; a user seeking recreational effects from a pill would likely restrict oral abuse to doubling, tripling, or quadrupling a dosage amount. However, utilizing apomorphine to prevent recreational use would inherently prevent the more dangerous harmful intentioned uses.

The overdose amount of a therapeutic agent includes an amount suited to cause unacceptable health risks to a user substantially disproportionate to the health benefits of the therapeutic agent. In some instances, the overdose amount of a therapeutic agent would be the LD₅₀ dose for the particular therapeutic agent; in other circumstances the overdose amount might be the amount of the therapeutic agent capable of causing a serious medical consequences, e.g. a coma or internal bleeding; or in other circumstances, the overdose amount might be the amount of the therapeutic agent capable of causing an irreversible medial event, e.g. a stroke, to the user. A preferred reference for obtaining an overdose amount is the Physicians' Desk Reference, from which the present invention may be formulated to combat any of the unsafe dosage circumstances (e.g. in relation to children, in relation to a general recommended dosage, in relation to pregnant women, etc.). Another preferred means for determining an overdose amount can be an integer multiple of the recommended dosage. The present invention includes dosages forms and systems tailored to the overdose amounts of the population at large, a subpopulation group, or adjusted to a specific individual having dependency problems.

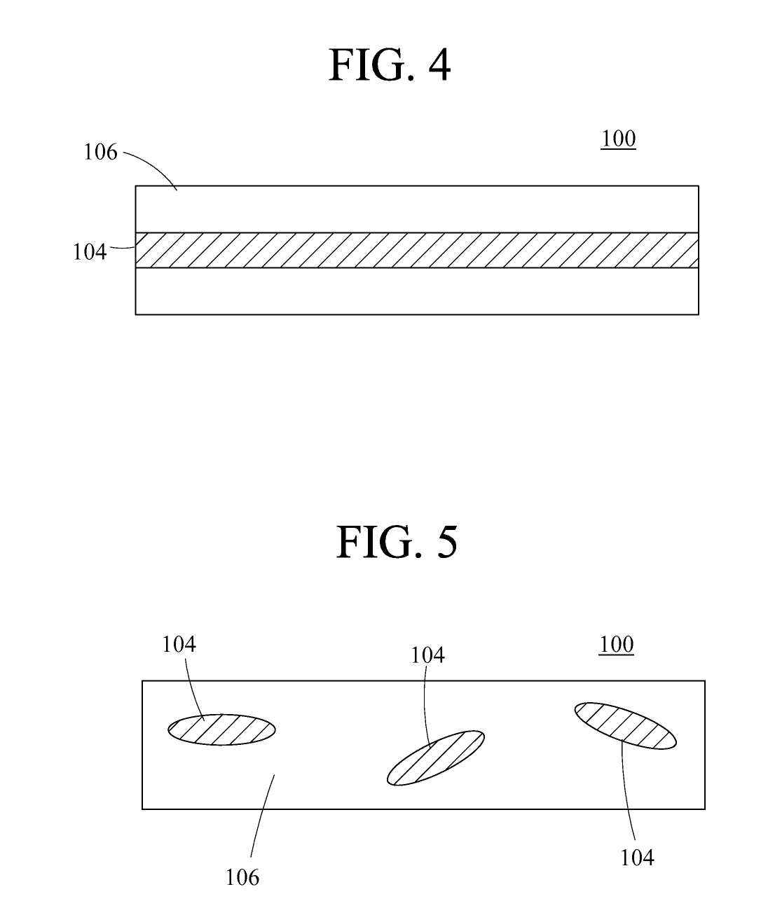

The apomorphine and the therapeutic agent need not be physically intermingled as illustrated by FIG. 2; as shown by FIGS. 4 and 5, the oral dosage form 100 of the present invention may include an apomorphine deposit 104 disbursed in a discrete layer within the oral dosage form 100, or the apomorphine deposit 104 may be disbursed in discrete pockets within the therapeutic agent. As apomorphine is generally not reactive, the apomorphine need not be separated from the therapeutic agent unless the chemical traits of the particular therapeutic agent require separation from apomorphine.

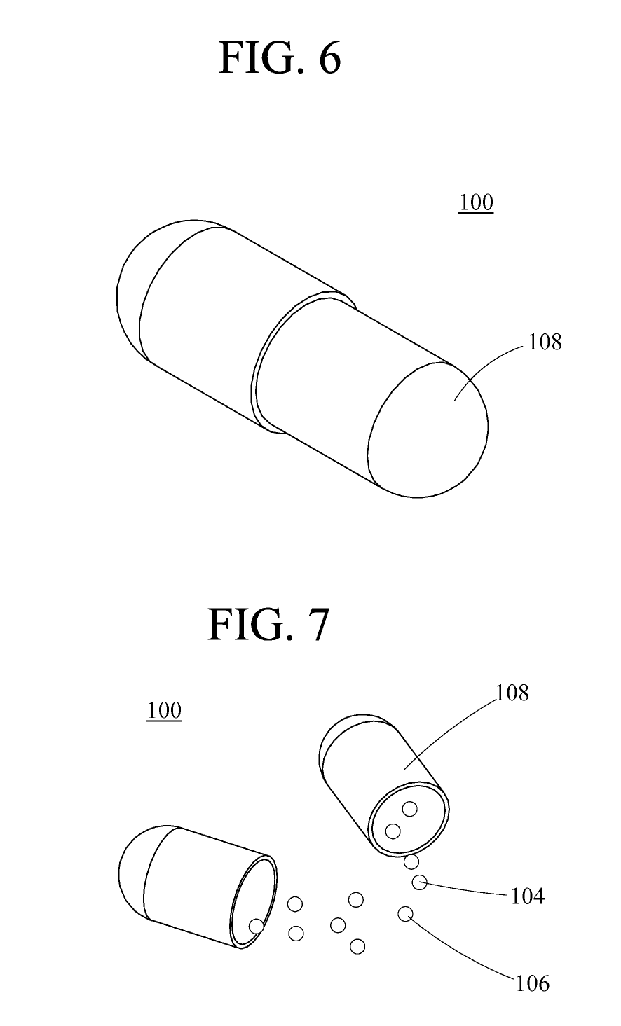

As shown by FIGS. 6 and 7, the oral dosage form 100 may include capsules. The contents of the capsules may include purposefully discrete portions of apomorphine and the therapeutic agent, or an intermingled powder combining apomorphine with the therapeutic agent. Any oral formulation of a drug, whether a tablet, caplet, capsule, or liquigel form, intended for oral ingestion is subsumed by the present invention. Furthermore, additions recognized in the oral dosage art may be combined with the present invention, e.g. suitable excipients, binders, and/or individual containing membrane (for capsules of liquigels) so as to provide for either an instant release or an extended release of drug after ingestion.

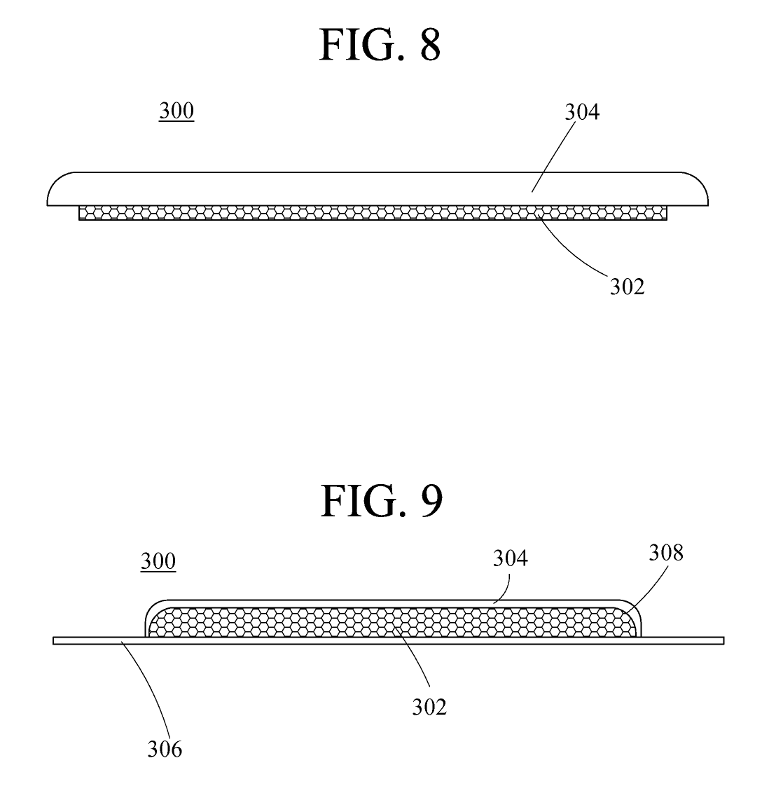

As shown by FIGS. 8 and 9 the present invention further includes a transdermal delivery system 300. The transdermal delivery system 300 includes a therapeutic composition stratum 302 and at least 50 mg of apomorphine disbursed within the stratum, or an amount of apomorphine adapted to allow a universal bioavailability of at least 2.0 mg of apomorphine. The transdermal delivery system may include a matrix transdermal patch, as shown in FIG. 8. The matrix transdermal patch includes a backing 304 having the therapeutic agent stratum 302 exposed for direct contact with the skin. The matrix patch embodiment may include other aspects related to common matrix patches, e.g. an adhesive mixed into, or immediately contacting, the therapeutic agent stratum. The apomorphine, due to its generally unreactive nature, is preferably directly disbursed within the therapeutic agent stratum 302. Distinct patch layers, or other segregation devices, may be used to separate the apomorphine from the therapeutic agent composition, but it is preferred that the apomorphine is intermingled with therapeutic agent composition to frustrate the efforts of users having the time and energy to disassemble a patch.

As FIG. 9 depicts, the transdermal delivery system 300 may further include a reservoir patch having a backing 302 in contact with an adhesive membrane 306. The reservoir patch transdermal delivery system includes a reservoir 308 housing the therapeutic agent 30 with an amount of apomorphine disbursed therein. The apomorphine, due to its generally unreactive nature, is preferably directly disbursed within the therapeutic agent composition 302. Distinct patch layers within the reservoir, or other segregation devices, may be used to separate the apomorphine from the therapeutic composition, but it is preferred that the apomorphine is intermingled with the therapeutic agent composition. The reservoir patch of the present invention may further include any other aspects related to common reservoir patches.

As transdermal delivery systems (i.e. “patches”) tend to be a preferred vehicle of the pharmaceutical industry for the safe delivery of large amounts of a therapeutic agent over long periods of time, transdermal patches will typically possess an amount of a therapeutic agent near the overdose amount. It is therefore preferred that each individual patch of the present invention include an amount of apomorphine disbursed within the therapeutic composition sufficient to allow a universal bioavailability of at least 2.0 mg of apomorphine.

The present invention further includes a nasal dosage system. The nasal dosage system includes a nasal dosage form, such as an aqueous nose spray, with a therapeutic agent. The therapeutic agent has a predetermined overdose amount, and the volume containing that predetermined overdose amount also preferably contains at least approximately 4.0 mg of apomorphine, or an amount of apomorphine adapted to generate an intra-nasal bioavailability of at least 2.0 mg of apomorphine. Unlike most embodiments of the present invention, it is not preferred that the nasal dosage system include apomorphine adapted to be universally bioavailable at an amount effective to cause emesis coupled to an overdose amount of therapeutic agent. As a general matter, apomorphine adapted to be bioavailable in amounts capable of causing emesis via oral delivery or sublingual delivery when coupled with an overdose amount of therapeutic agent, could interfere with a proper administration regimen of the therapeutic agent intra-nasally. The primary purpose in using a proper of amount of apomorphine in the nasal dosage system is to prevent abuse via intra-nasal abuse or injection diversion.

In a topical cream embodiment of the present invention, a transdermal dosage form, e.g. a cream or gel, includes a therapeutic agent having a predetermined overdose amount. The volume of the transdermal dosage form containing the predetermined overdose amount also preferably includes at least 50 mg of apomorphine disbursed therein, or an amount of apomorphine adapted to allow a universal bioavailability of at least 2.0 mg of apomorphine. Other preferred topical cream embodiments are adapted to allow a universal bioavailability of 2.5 mg, 3.0 mg, or higher amounts.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. An oral dosage form comprising:
an oral carrier possessing an amount of a therapeutic agent, with a predetermined oral overdose amount, disbursed within said oral carrier; wherein a ratio of said therapeutic amount to said predetermined oral overdose amount is substantially less than or equal to a ratio of said apomorphine amount to an apomorphine security amount of at least 50.0 mg.

2. The dosage form of claim 1 wherein said oral carrier possesses an amount of apomorphine greater than 10.0 mg.

3. The dosage form of claim 2 wherein said oral carrier possesses an amount of apomorphine greater than 12.5 mg.

4. The dosage form of claim 1 wherein said apomorphine security amount is at least 62.5 mg.

5. The dosage form of claim 2 wherein said apomorphine security amount is at least 75.0 mg.

6. An oral dosage system comprising:
at least two oral dosage forms possessing a predetermined overdose amount of a therapeutic agent; and an amount of apomorphine, disbursed within said oral dosage forms, adapted to have a universal bioavailability of at least 2.0 mg of apomorphine at said predetermined overdose amount.

7. The oral dosage system of claim 6 wherein said amount of apomorphine is adapted to have a universal bioavailability of at least 2.5 mg.

8. The oral dosage system of claim 7 wherein said amount of apomorphine is adapted to have a universal bioavailability of at least 3.0 mg.

9. An oral dosage form comprising:
an oral carrier possessing an amount of a therapeutic agent, capable of substantial oral abuse, with a predetermined oral overdose amount; and apomorphine, disbursed within said oral carrier, present in an amount greater than 10 mg and sufficient to have a universal bioavailability of at least 2.0 mg when multiplied by a ratio of said predetermined overdose amount to said therapeutic amount.

10. The dosage form of claim 9 wherein said apomorphine is present in amount to have a universal bioavailability of at least 2.5 mg when multiplied by a ratio of said predetermined overdose amount to said therapeutic amount.

11. The dosage form of claim 10 wherein said apomorphine is present in amount to have a universal bioavailability of at least 3.0 mg when multiplied by a ratio of said predetermined overdose amount to said therapeutic amount.

12. An oral dosage system comprising:
at least two oral dosage forms having a therapeutic agent of a predetermined overdose amount; and at least 50.0 mg of apomorphine disbursed within a number of said oral dosage forms capable of allowing said predetermined overdose amount.

13. The oral dosage system of claim 12 comprising at least 62.5 mg of apomorphine.

14. The oral dosage system of claim 13 comprising at least 75.0 mg of apomorphine.

15. An oral dosage form comprising an oral carrier possessing a therapeutic agent and at least 10.0 mg of apomorphine.

16. The oral dosage form of claim 15 possessing at least 15.0 mg of apomorphine.

17. The oral dosage form of claim 16 possessing at least 20.0 mg of apomorphine.

18. The oral dosage form of claim 17 possessing at least 25.0 mg of apomorphine.

19. A transdermal delivery system comprising:
a therapeutic composition stratum;
an amount of apomorphine, disbursed within said therapeutic composition stratum, adapted to allow a universal bioavailability of at least 2.0 mg of apomorphine.

20. The transdermal delivery system of claim 19 comprising an amount of apomorphine adapted to allow a universal bioavailability of at least 2.5 mg of apomorphine.

21. The transdermal delivery system of claim 20 comprising an amount of apomorphine adapted to allow a universal bioavailability of at least 3.0 mg of apomorphine.

22. A transdermal delivery system comprising:
a therapeutic composition stratum;
at least 50.0 mg of apomorphine substantially uniformly disbursed within said therapeutic composition stratum.

23. The transdermal delivery system of claim 22 comprising at least 62.5 mg of apomorphine.

24. The transdermal delivery system of claim 23 comprising at least 75.0 mg of apomorphine.

25. A nasal dosage system comprising:
a nasal dosage form having a therapeutic agent of a predetermined overdose volume, and at least 2.0 mg of apomorphine disbursed within said predetermined overdose volume.

26. The nasal dosage system of claim 25 comprising at least 3.0 mg of apomorphine.

27. The nasal dosage system of claim 26 comprising at least 4.0 mg of apomorphine.

28. A nasal dosage system comprising:
a nasal dosage form having a therapeutic agent of a predetermined overdose volume, and an amount of apomorphine within said predetermined overdose volume adapted to allow an intranasal bioavailability of at least 1.0 mg of apomorphine.

29. The nasal dosage system of claim 28 wherein said amount of apomorphine is adapted to have an intranasal bioavailability of at least 1.5 mg.

30. The nasal dosage system of claim 29 wherein said amount of apomorphine is adapted to have an intranasal bioavailability of at least 2.0 mg.

31. A topical cream comprising:
a transdermal dosage cream having a therapeutic agent of a predetermined overdose volume, and at least 50.0 mg of apomorphine disbursed within said predetermined overdose volume.

32. The topical cream of claim 31 comprising at least 62.5 mg of apomorphine.

33. The topical cream of claim 32 comprising at least 75.0 mg of apomorphine.
